(19) United States
(12) Patent Application Publication
(10) Pub. No.: US 2002/0071869 A1
(43) Pub. Date: Jun. 13, 2002

(54) Microparticle composition and method

(76) Inventors: Petr Bures, Coraopolis, PA (US); David B. Henthorn, Lafayette, IN (US); Nicholas A. Peppas, Indianapolis, IN (US)

Correspondence Address: BARNES & THORNBURG, 11 SOUTH MERIDIAN, INDIANAPOLIS, IN, 46204

(21) Appl. No.: 09/935,379

(22) Filed: Aug. 22, 2001

Related U.S. Application Data
(60) Provisional application No. 60/226,813, filed on Aug. 22, 2000.

(51) Int. Cl. A61K 9/14; C08F 118/02

(52) U.S. Cl. 424/487; 526/319

(57) ABSTRACT

The invention relates to a cross-linked hydrogel composition in the form of substantially uniform microparticles and a method of preparation therefor. The hydrogel composition comprises a crosslinked polymer formed by free radical polymerization of olefin monomers comprising a C₃-C₆ unsaturated carboxylic acid and a water dispersible polyolefin crosslinking agent. The olefin monomers may further comprise a polyalkyleneglycol monoacrylate or momomethacrylate.

### CROSS REFERENCE TO RELATED APPLICATIONS

This application claims priority under 35 U.S.C. § 119(e) to U.S. Provisional Application No. 60/226,813, filed Aug. 22, 2000.

### GOVERNMENT RIGHTS

Research relating to this invention was supported in part by the U.S. Government under Grant No. GM56231 awarded from the National Institutes of Health. The U.S. Government may have certain rights in this invention.

### FIELD OF THE INVENTION

The present invention relates to a hydrogel composition in the form of microparticles and a method of preparation therefor. More particularly, the invention relates to a particulate hydrogel composition comprising a C₃-C₆ unsaturated carboxylic acid and a polyolefin crosslinking agent.

### BACKGROUND AND SUMMARY OF THE INVENTION

The development of methods for generating new drug candidates using combinatorial chemistry techniques and the development of solid-phase peptide synthesis and of recombinant DNA technology has stimulated interest in the potential for using new drugs, including small molecules, proteins, and peptides, as therapeutic agents. Accordingly, there has been a significant research and development effort directed to the synthesis and testing of new drugs for treating various disease states. While there has been rapid progress in the synthesis of new drugs, progress in the development of formulations and delivery systems is lacking.

One vehicle for use in drug delivery is polymeric microspheres with the drug dispersed in the polymeric matrix for use in controlled release of the drug. Such microspheres may have a diameter ranging from about 1 to about 1000 μm. When administered in vivo, the drug diffuses out of the microsphere and its release rate is controlled by the type of polymer used in the microsphere. Microspheres loaded with drug may be administered orally, subcutaneously, or intravenously.

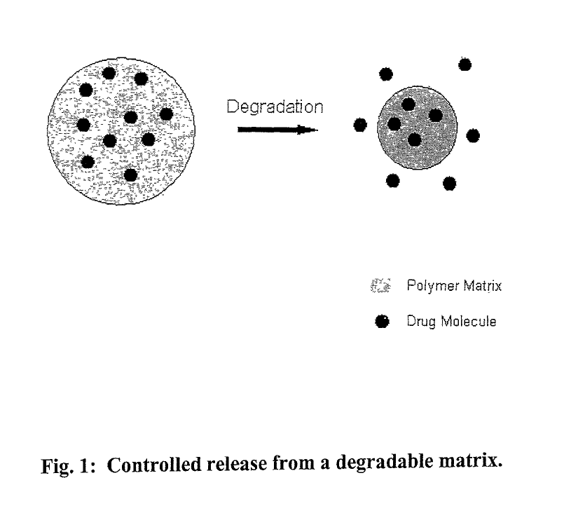

Microspheres used in the controlled release of drugs are of two categories. Biodegradable microspheres are made from water insoluble polymers that degrade in vivo, either by hydrolysis or by enzymatic degradation. Copolymers of poly(lactic acid) and poly(glycolic acid) have received considerable attention due to their ability to hydrolyze into soluble products found naturally in the body. As the matrix degrades, drug dispersed in the matrix is released (FIG. 1). Degradation rates may be controlled by varying the ratio of glycolic acid to lactic acid repeating units in the copolymer.

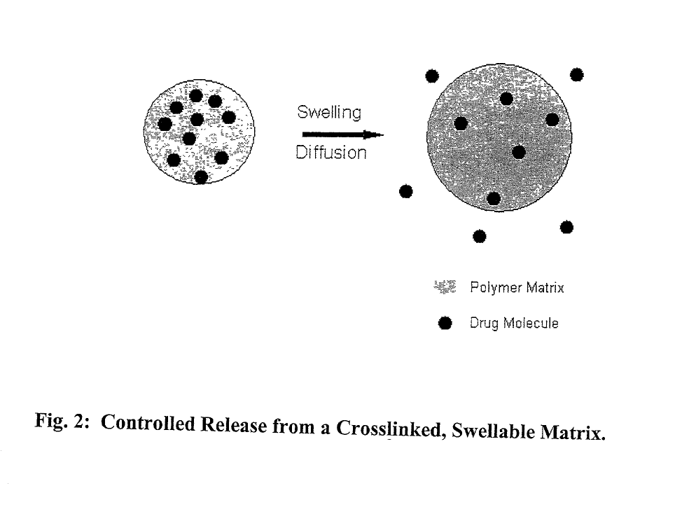

Hydrophilic microspheres (i.e., hydrogels in the form of microspheres) may also be used for controlled drug delivery. Hydrogels are three dimensional, crosslinked networks that swell in compatible solvents. Hydrogels have been used in controlled drug delivery because they are non-toxic and the release of drug may be easily mediated by changing the morphology of the gel. Dissolution of the hydrogel is prevented by crosslinks, which can be either physical, such as chain entanglements, or chemical. The volume swelling ratio, Q, is defined as:

$$Q = \frac{V}{{V}_{0}}$$

where V is the volume of the gel and V₀ is the initial volume of the dried gel, and is the most important characteristic of a hydrogel. Determination of the crosslinking density is done by either allowing the gel to swell to equilibrium and measuring the equilibrium volume swelling ratio or by measuring the equilibrium elastic modulus as determined by mechanical testing. These particles differ from hydrophobic matrices in that release of drug is facilitated by the solvent uptake and swelling processes (FIG. 2).

Surface characteristics and particle size are two important parameters in microsphere synthesis because these parameters influence the capacity of microspheres to be delivered to a specific site. Desired microsphere size is dependent on the administration site used. For example, for intravenous administration, microspheres must be small enough to prevent capillary occlusion. Microspheres for subcutaneous use may be much larger.

Numerous methods exist for the production of microspheres. Microsphere preparation will ideally will be a technique that:

(i.) maximizes drug loading;

(ii.) minimizes exposure of drugs to denaturing conditions during loading;

(iii.) is rapid and reproducible; and

(iv.) produces nearly monodisperse microspheres of the size needed for delivery to a specific site.

The simplest method for production of microspheres is the grinding of a solid substrate into micron-sized particles. Other methods focus on the production of spherical particles during microsphere synthesis. One such method is solvent evaporation, a technique commonly employed in the preparation of biodegradable polymer microspheres. A polymer is dissolved in a suitable solvent and then dispersed in an agitated continuous phase containing a nonsolvent and a suspending agent. Over time, the solvent evaporates leaving solid polymeric microspheres.

Microspheres may also be formed during polymerization, such as by methods including emulsion and suspension polymerization. In suspension polymerization, monomer and initiator are dispersed in an agitated continuous phase containing a suspending agent. Droplets are formed that are sensitive to such parameters as rate of agitation, the viscosities of the dispersed and the continuous phases, and surfactant interaction. Polymerization of the monomer droplets forms solid polymer spheres that may be collected by centrifugation or filtration.

Emulsion polymerization involves the dispersion of monomers in a continuous phase containing an emulsifying agent. Micelles are formed by the emulsifying agent which swell with the monomers and form solid particles. In emulsion polymerization, the micelle size ultimately governs the final particle size.

Dispersion polymerization, applicable to a limited number of systems, is another method of microsphere formation. Monomer and initiator are dissolved in a solvent which dissolves the monomer but acts as a nonsolvent to the resulting polymer. Because of the insolubility of the polymer in the solvent, growing polymer chains aggregate and form a second phase.

Initiators are typically used to produce polymeric microspheres. Initiation of free radical polymerization reactions is traditionally done using a thermal initiator. A typical water soluble thermal initiator is 2,2′-azobis isobutyronitrile (AIBN) which is used at reaction temperatures of 50-90° C. At these temperatures proteins may denature, rendering them inactive. Redox initiators, such as sodium metabisulfite and ammonium persulfate, have been used in the production of polymer gels and may be used at lower temperatures. One method that has received only moderate attention is production of microspheres by a suspension or emulsion polymerization initiated by ultraviolet (UV) light. UV initiation is rapid and easily controlled. However, in suspensions and emulsions, the phase separation scatters light and limits the penetration depth of the incident radiation.

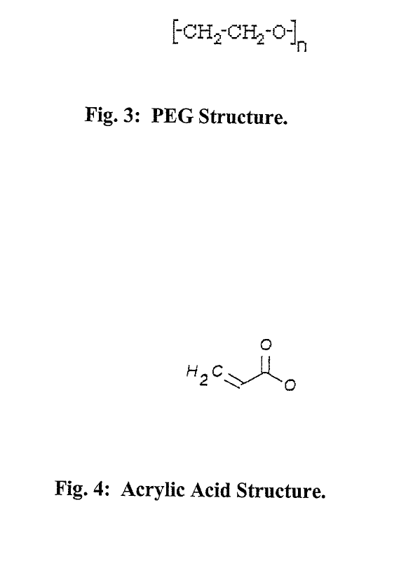

Poly(ethylene glycol) (PEG) (FIG. 3) is a widely used biomedical polymer because of its unique properties when in contact with biological fluids including hydrophilicity, mechanical stability, thermal stability, and stealth properties in the reticulo-endothelial system. PEG has also been shown to act as a bioadhesion promoter when incorporated into gels of poly(acrylic acid) (PAA) (FIG. 4).

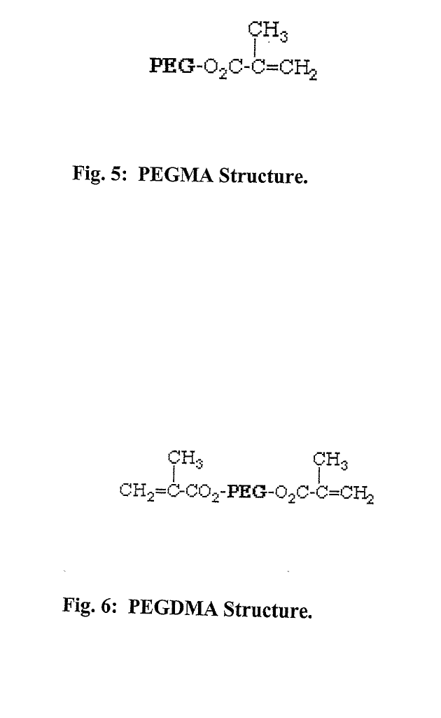

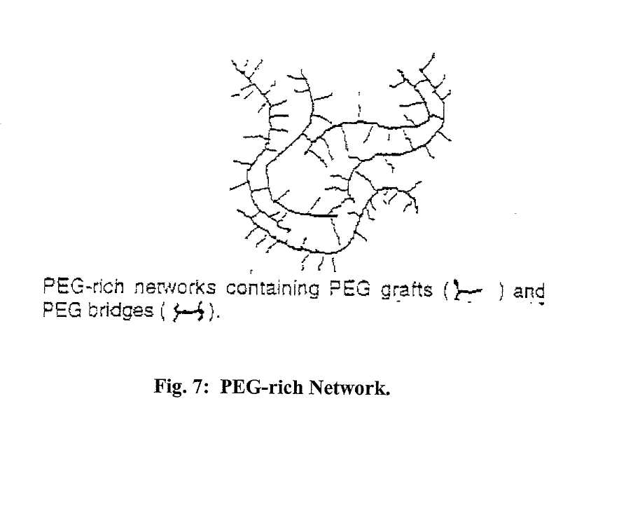

PEG has been copolymerized with the macromonomers poly(ethylene glycol) monomethacrylate (PEGMA) (FIG. 5) and poly(ethylene glycol) dimethacrylate (PEGDMA) (FIG. 6). Free radical polymerization initiated by UV light formed a methacrylate backbone with grafted PEG chains and PEG bridges acting as crosslinks (FIG. 7). The PEGMA and PEGDMA macromonomers had PEG side chain molecular weights of 1000. Gels with higher amounts of PEGDMA swelled less than those prepared with higher amounts of PEGMA, and release of diltiazem (MW 451) from the gel was shown to be a function of the PEGDMA to PEGMA ratio with the release rate increasing with decreasing amounts of PEGDMA.

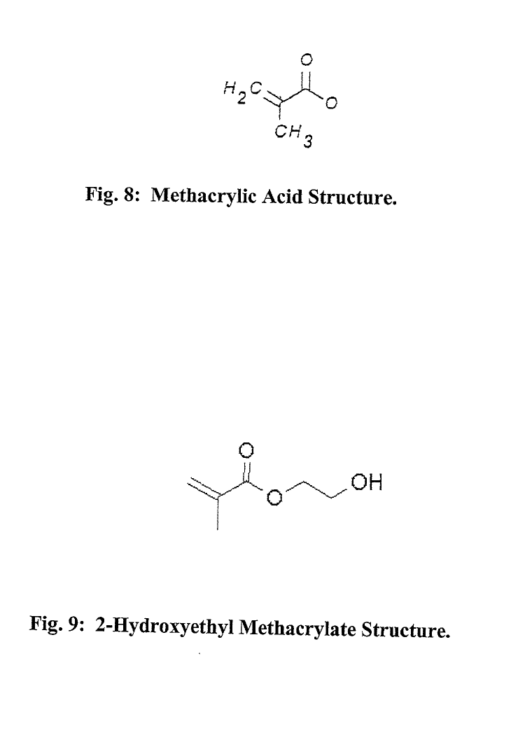

Graft copolymers of methacrylic acid (MAA) (FIG. 8) and PEG have also been formed. Under acidic conditions, the PMAA backbone is protonated and may form an intermolecular complex with grafted PEG chains. Gels of P(MAA-g-EG) have a higher of swelling at higher pH's with the smallest degree of swelling shown by gels with MAA to EG repeating unit ratios of 1:1. Thus, gels of P(MAA-g-EG) have potential use as matrices for controlled release that is responsive to pH changes. Recent applications include the controlled release of insulin either orally or from a glucose responsive hydrogel.

Microspheres of P(MAA-g-EG) have been prepared by a suspension polymerization reaction initiated thermally. Silicon oil was used as the continuous phase with poly(dimethylsiloxane-b-ethylene oxide) added as a surfactant. The polymerization reaction was conducted first at 70° C. followed by a period at 90° C. Particle size was dependent on surfactant concentration and ranged from 18 to 30 μm and the MAA to EG ratio was 11.6 MAA repeating units for every EG repeating unit.

Polymers of 2-hydroxyethyl methacrylate (HEMA) (FIG. 9) have been prepared and are known to exhibit good biocompatibility and to form hydrogels with good swelling and release characteristics. P(HEMA) microspheres have been produced using an all-aqueous system. When dissolved in an aqueous NaCl solution HEMA separates into an aqueous phase rich in HEMA and an aqueous phase that is predominately salt. HEMA, ethylene glycol dimethacrylate (EGDMA), and thermal initiator were dissolved in a NaCl solution with Mg(OH)₂ added as a stabilizer. Agitation produced dispersed droplets of monomer and upon polymerization solid microspheres were formed that were approximately 100 μm and in diameter were polydisperse.

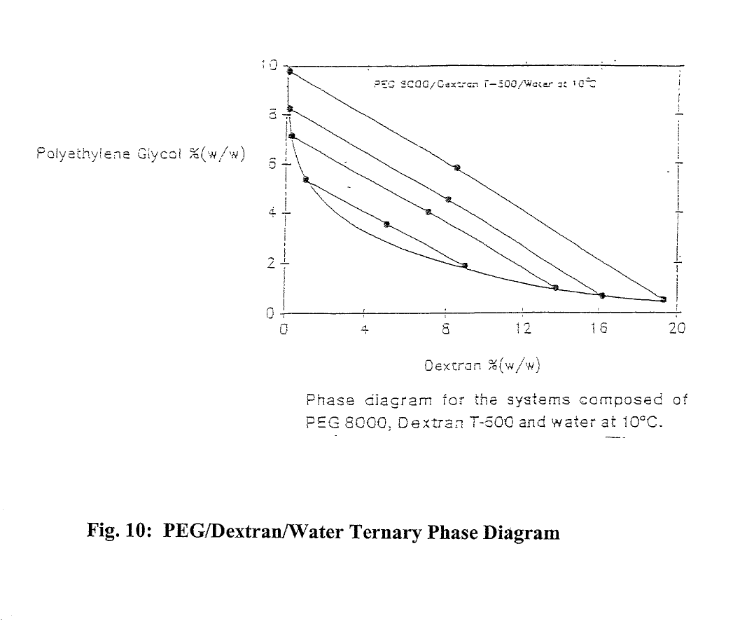

It is known that many ternary systems of two polymers in a mutual solvent exhibit phase separation in solution. In the bioseparations field, one such system that has received significant attention is aqueous PEG/dextran in water. Dextran is a branched polysaccharide consisting of α-(1→4) and α-(1→6) linked glucose residues that has a long history of use as a biomaterial, including use as a blood plasma extender since the 1940's. Currently, the PEG/dextran/water ternary system for use in protein separations is being studied. Certain dissolved proteins partition to the slightly more hydrophobic PEG phase leaving behind impurities in the more hydrophilic dextran phase. Considerable thermodynamic data (FIG. 10) has been collected on the PEG/dextran systems and may be used to predict phase separation. Enzymatically degradable microparticles of dextran have been prepared by an all-aqueous emulsion polymerization. Methacrylated dextran was suspended in an aqueous PEG continuous phase and agitated to form dispersed droplets. After polymerization, the microparticles were centrifuged and washed prior to release experiments. Mean particle diameter was determined to be 10 μm.

Accordingly, microsphere compositions have been produced for the controlled release of drugs. However, microsphere compositions with improved swelling and biocompatibility characteristics are needed, and a method for producing microspheres that are non-toxic for use in pharmaceutical and biomedical applications is needed (i.e., a method for producing microspheres in the absence of organic solvents). Thus, the focus of the present invention was to develop a method for the preparation of polymeric microspheres for use as a controlled drug release matrix. The microspheres of the present invention may contain PEG, a polymer that is known for its unique swelling properties in vivo and for its biocompatibility.

In one embodiment, a method of forming a crosslinked hydrogel in the form of substantially uniform microparticles is provided. The method comprises polymerizing a monomer composition comprising a C₃-C₆ unsaturated carboxylic acid and a polyolefin crosslinking agent dispersed in an aqueous medium at a pH of less than 4.5. In another embodiment, the monomer composition further comprises a polyalkylene glycol monoacrylate or monomethacrylate.

In yet another embodiment, a crosslinked hydrogel composition in the form of substantially uniform microparticles is provided. The hydrogel composition comprises a crosslinked polymer formed by free radical polymerization of olefin monomers comprising a C₃-C₆ unsaturated carboxylic acid and a water dispersible polyolefin crosslinking agent in an aqueous medium at a pH of less than about 4.5. In still another embodiment the carboxylic acid is acrylic or methacrylic acid. In a further embodiment the olefin monomers further comprise a polyalkyleneglycol monoacrylate or monomethacrylate.

### DETAILED DESCRIPTION OF THE INVENTION

The present invention is directed to hydrogel compositions in the form of substantially uniform microparticles and to a method of their preparation. The hydrogel compositions of the present invention may be used for delivering biologically active proteins and drugs to vertebrates, preferably via oral administration. For example, in accordance with one embodiment the orally administered composition comprises a swellable hydrogel, and a drug contained within the swellable hydrogel for controlled release of the drug. Other uses are contemplated, however, and include use of the hydrogel compositions of the present invention as flocculents aiding flocculation in waste water and other industrial fluids, as carriers in cosmetic applications (e.g., for creams, essential oils, perfumes, etc.), as superabsorbent materials (e.g., for use in diapers, incontinence products, feminine hygiene products, etc.), and as chromatographic packing materials.

The term “hydrogel” as used herein generally refers to a synthetic polymer which is capable of taking up water by hydration.

The term “microparticle” in accordance with the present invention is defined as a substantially spherical particle having a diameter in the range of about 10 nm to about 1 mm. In one embodiment, the microparticles of the present invention have an average diameter of about 1 to about 1000 microns. In another embodiment, the average diameter of the microparticles is about 0.1 to about 500 microns. The microparticles produced in accordance with the method of the invention are substantially uniform in size and shape.

The term “drug” in accordance with the present invention means any substance/compound which induces a desired local or systemic effect in a patient in need of such an effect.

Hydrogels are water swellable, cross-linked polymer matrices that are well known to those of ordinary skill in the art. See, for example, Dresback, U.S. Pat. No. 4,220,152, issued Sep. 2, 1980, the disclosure of which is expressly incorporated herein by reference. Hydrogels have been found to be an effective delivery vehicle for orally delivering proteins to vertebrate species. The swellable properties of hydrogels can be utilized, first to protect the hydrogel contents from the harsh environments of the stomach as the composition passes through the digestive tract, and then to release the hydrogel contents into the more favorable regions of the GI tract, specifically the lower regions of the intestine. Thus, the hydrogel composition can be used as an efficacious drug delivery system, particularly for proteins or peptides which are stabilized by entrapment in the hydrogel.

Hydrogels can be impregnated or loaded with a variety of bioactive compounds, including but not limited to drugs, growth hormones, vaccine compositions, vitamins, steroids and peptides, and used as a delivery vehicle for orally administering such compounds. Compounds loaded into the hydrogel are released in a controlled manner as the hydrogel becomes hydrated within the animal's digestive system. In one embodiment, the present hydrogel matrix is in a pelletized form comprised of polymethacrylic acid, and the polymethacrylic acid polymers are grafted with an ionic long chain polymer such as polyethylene glycol (PEG).

In accordance with one embodiment the hydrogels are loaded with a drug by equilibrium portioning. More particularly, the hydrogels are hydrated in a solution having a pH of greater than about 5.8 and containing the composition to be loaded. The hydrogels are then recovered and washed with a solution having a pH of less than about 5.8 and the loaded hydrogels are then dried and stored at 4° C. Another method of loading the hydrogels of the present invention comprises the steps of adding an aqueous solution of the desired compound to a solution of monomers and a cross-linker, and initiating polymerization of the mixture.

Any drug may be delivered via the hydrogel compositions of the present invention. The drug for use in accordance with the hydrogel compositions of the invention can be a non-protein drug or a protein, a peptide, or a peptidomimetic. The hydrogel compositions in accordance with the present invention stabilize proteins and peptides resulting in increased bioavailability, and, thus, are particularly advantageous for the delivery of proteins and peptides. However, any active drug which is physically and chemically compatible with the polymers that comprise the present hydrogel compositions may be used. The drug, upon addition to the hydrogel composition, may comprise from about 1 weight percent to about 60 weight percent of the hydrogel composition depending on the loading capacity of the hydrogel relative to the drug and the dose of drug desired.

Representative drugs which can be utilized in the present invention include, but are not limited to, antiarthritis agents, antacids, anti-gout agents, antiviral agents, anti-protozoal agents, adrenergic blocking agents, anti-infectives, antihypertensive agents, analgesics, adrenal cortical steroid inhibitors, anti-inflammatory agents, antiarrhythmics, sedatives, vasodilators, psychotropics, vitamins, antihistamines, anti-obesity drugs, antiemetics, antianginal agents, vasoconstrictors, drugs used to treat migraine headaches, antipyretics, hyper- and hypoglycemic agents, diuretics, anti-nauseants, anticovulsants, mucolytics, neuromuscular drugs, anabolic drugs, antispasmodics, diuretics, antiasthmatics, hormones, and uterine relaxants.

Exemplary of proteins, peptides, and peptidomimetics that can be used in accordance with the present invention are TRH, desmopressin acetate, LHRH agonists, D-Ala, D-Leu-enkephalin, metkephamid, oxytocin, insulin-like growth factors, growth hormone releasing hormone, sleep inducing peptide, opiate antagonists, opiate agonists, somatostatin, peptide T, vasoactive intestinal polypeptide, gastric inhibitory peptide, cholecystokin and its active fragments, gastrin releasing peptide, ACTH and its analogs, enkephalins, growth hormones, interferons, interleukins, calcitonin, insulin-like growth factors, insulin, colony stimulating factor, tumor inhibitory factors, transforming growth factors, epidermal growth factor, atrial naturetic factor, proinsulin, nerve growth factor, transforming growth factor beta, and glucagon.

The hydrogel pellets are preferably synthesized by polymerizing methacrylic acid, in the presence of a crosslinking agent. However, the monomer composition for use in accordance with the invention comprises a C₃-C₆ unsaturated carboxylic acid and the C₃-C₆ unsaturated carboxylic acid may be any C₃-C₆ unsaturated carboxylic acid including an acrylic acid or a methacrylic acid.

The crosslinking agent may be di-olefin functional or tri-olefin functional. The crosslinking agent can be selected from a wide variety of biocompatible crosslinking agents known to those skilled in the art such as a polyalkyleneglycol diacrylate or dimethacrylate. Crosslinking agents further include tetraethylene glycol dimethacrylate, ethylene dimethacrylate, diethylene dimethacrylate, triethylene dimethacrylate, tetraethylene dimethacrylate, pentaethylene dimethacrylate, the corresponding diacrylates, or a star polymer comprising methacrylate, acrylate or methylene bis-acrylamido groups.

Polymerization is initiated with a free radical initiator such as thermal initiators including organic peroxides or UV radical initiators known to those skilled in the art. Thermal initiators for use in synthesis of the hydrogel compositions of the present invention include any water soluble thermal initiator known in the art. Exemplary of such thermal initiators are peresters, peroxycarbonates, peroxides, azonitrile compounds, and the like, including such thermal initiators as ammonium persulfate, benzoyl peroxide, 2,2-azobis (2-methylpropionamidine) dihydrochloride, and azobis(isobutyronitrile). Additionally, any UV initiators known in the art may be used to promote the polymerization reaction of the present invention. Exemplary of such UV initiators are benzoin alkyl ethers, benzophenone, camphorquinone, Darocur 1173, Irgacure 184, 2,2-Dimethoxy-2-phenylacetophenone (DMPA) and the like. Polymerization may also be initiated by γ-radiation or electron beam radiation.

The aqueous medium of a pH of less than about 4.5 in which the monomer composition of the invention is dispersed may also contain a cationic, anionic, or nonionic surfactant. Such surfactants include sodium dodecyl sulfate, Tween® 80 (poly(oxyethylene) sorbitan ester), and Span® 80 (sorbitan monooleate), but any suitable surfactant known the art may be used.

Suitable olefin functional polyalkylene glycol monomers which may further be present in the monomer composition for forming the hydrogels of the present invention include, but are not limited to, acrylates, methacrylates, and esters of polyalkylene glycols, for example, polyethylene glycol (PEG) and polypropylene glycol (PPG), or combinations thereof. Exemplary of such compounds are compounds of formula:

R₁O—R₂—OR₃

wherein

R₁=(meth)acrylate;

R₂=poly(C₂-C₄ alkylene) glycol;

and R₃=H, C₁-C₄ alkyl, C₁-C₄ alkanoyl, acrylate or methacrylate.

The molecular weight of such olefin functional monomers is typically about 200 to about 2500, but may be between about 200 to about 4500 or between about 200 to about 18,000.

In one embodiment the hydrogel matrix comprises a co-polymer of methacrylic acid and a poly(alkylene glycol) monomethacrylate (or monoacrylate) crosslinked with a biocompatible crosslinking agent. “Poly(alkylene glycol) monomethacrylate” as used herein includes poly(ethylene glycol) monomethacrylate, poly(propylene glycol) monomethacrylate and poly(ethylene/propylene glycol) monomethacrylate, wherein poly(ethylene/propylene glycol) monomethacrylate is the polymer formed by hydroxy functional methacrylate initiated polymerization of a mixture of ethylene oxide and propylene oxide. The resulting pendant poly(alkylene glycol) groups have a molecular weight ranging from about 200 to about 4000, more typically about 200 to about 2000, and in one embodiment about 200 to about 1200. The molar ratio of methacrylic acid and poly(alkylene glycol) monomethacrylate (or monoacrylate) monomers is about 4:1 to about 1:4.

The particulate hydrogel composition in accordance with the present invention can be used for administration to a patient and can be in an unencapsulated form, for example, suspended or dispersed in a liquid or solid carrier, or in an encapsulated form such as a capsule for oral administration or a microparticle, for example, one having a single layer comprising the hydrogel composition and an outer skin layer. These are versatile drug delivery systems that can be used for delivering single drugs in single doses, or they can be used to deliver multiple drugs in simultaneous doses.

Pharmaceutical compositions comprising the hydrogel composition of the present invention in unencapsulated form or in an encapsulated form are also provided. The hydrogel composition in unencapsulated form may be used to deliver a dose of a drug, or a combination of drugs, in a prolonged release dose.

Pharmaceutical compositions may be used for oral ingestion in the form of a tablet, a capsule, a caplet, a gel-seal, a lozenge, liquid dosage forms such as syrups, sprays, and other liquid dosage forms, or any other dosage form useful for drug delivery using the hydrogel composition of the present invention. Buccal and sublingual administration comprises contacting the oral and pharyngeal mucosa of the patient with the dose of unencapsulated or encapsulated hydrogel composition either in a pharmaceutically acceptable liquid dosage form, such as a syrup or a spray, or in a saliva-soluble dosage form which is held in the patient's mouth to form a saliva solution of the drug in contact with the oral and pharyngeal mucosa. Exemplary of saliva-soluble dosage forms are lozenges, tablets, and the like. The unencapsulated or encapsulated forms of the hydrogel composition may also be administered parenterally in the form of a liquid solution, such as in the form of a suspension in a pharmaceutically acceptable buffer. Such parenteral administration may be intradermal, subcutaneous, intramuscular, intraperitoneal, intravenous, or the like.

Syrups may be flavored or unflavored and may be formulated using a buffered aqueous solution of unencapsulated or encapsulated forms of the particulate hydrogel composition as a base with added caloric or non-caloric sweeteners, flavor oils and pharmaceutically acceptable surfactant/dispersants. Other liquid dosage forms, including solutions or sprays, can be prepared in a similar manner and can be administered buccally, sublingually, or by oral ingestion.

Tablets for use in accordance with this invention can be prepared by art-recognized techniques for forming compressed tablets where the unencapsulated or encapsulated forms of the particulate hydrogel composition are dispersed on a compressible solid carrier, optionally combined with any appropriate tableting aids such as a lubricant (e.g., magnesium-stearate) and are compressed into tablets, or by other art-recognized techniques for forming compressed tablets such as chewable vitamins. Suitable solid carrier components for tableting include manitol, microcrystalline cellulose, carboxymethyl cellulose, and dibasic calcium phosphate.

Solid dosage forms for oral ingestion administration include such dosage forms as lozenges, caplets, capsules, and gel-seals. Such solid dosage forms can be prepared using standard tableting protocols and excipients to provide lozenges, caplets, capsules, or gel-seals containing unencapsulated or encapsulated forms of the hydrogel composition.

A “pharmaceutical acceptable carrier” for use in accordance with the invention is compatible with other reagents in the pharmaceutical composition and is not deleterious to the patient. The pharmaceutically acceptable carrier formulations for pharmaceutical compositions adapted for oral ingestion or buccal/sublingual administration including lozenges, tablets, capsules, caplets, gel-seals, and liquid dosage forms, such as syrups, sprays, and other liquid dosage forms, have been described above. Unencapsulated or encapsulated forms of the hydrogel composition can also be adapted for parenteral administration in accordance with this invention using a pharmaceutical acceptable carrier adapted for use in a liquid dose form. Thus, unencapsulated or encapsulated forms of the hydrogel composition can be administered dissolved in a buffered aqueous solution. Such a liquid solution may be in the form of a suspension. Exemplary of a buffered solution suitable as a carrier in accordance with the present invention for unencapsulated or encapsulated forms of the hydrogel composition administered parenterally is phosphate buffered saline prepared as follows:

A concentrated (20×) solution of phosphate buffered saline (PBS) is prepared by dissolving the following reagents in sufficient water to make 1,000 ml of solution: sodium chloride, 160 grams; potassium chloride, 4.0 grams; sodium hydrogen phosphate, 23 grams; potassium dihydrogen phosphate, 4.0 grams; and optionally phenol red powder, 0.4 grams. The solution is sterilized by autoclaving at 15 pounds of pressure for 15 minutes and is then diluted with additional water to a single strength concentration prior to use.

The daily doses of unencapsulated or encapsulated forms of the hydrogel composition for administration in accordance with this invention can be administered as single doses, or they can be divided and administered as a multiple-dose daily regimen. Thus, the doses may be administered 1 to 4 times a day until patient symptoms have subsided or are stabilized. Further, a staggered regimen, for example, one to three days of treatments per week, can be used as an alternative to daily treatment, and for the purpose of defining this invention such intermittent or staggered daily regimen is considered to be equivalent to every day treatment and within the scope of this invention.

### EXAMPLE 1

### Preparation Of Microparticles

Preparation of Poly(ethylene Glycol Dimethacrylate) Microparticles by Suspension Polymerization.

Microparticles of PEGDMA were prepared by a free radical polymerization initiated by UV light (EFOS Ultracure 100, Buffalo, N.Y.). PEGDMA (250 mg) (Polysciences, Inc., Warrington, Pa.), having a PEG chain molecular weight of 600 Daltons, was dissolved in 1 g distilled water along with 10 mg 2,2-Dimethoxy-2phenylacetophenone (DMPA) (Aldrich, Milwaukee, Wis.) as a UV initiator. The resulting monomer solution was dispersed in 25 g silicon oil (Dow Chemical) containing 1% (wt/wt) sorbitan monooleate (Aldrich).

The two-phase mixture was irradiated with UV light (1 mW/cm²) for 15 minutes at 25° C. The mixture was stirred during polymerization at 300 rpm with a 1″ magnetic stirring bar. The resulting particles were twice centrifuged (3,000× g) for 15 minutes and resuspended in hexane. Finally, the particles were dried in a vacuum oven for 24 hours.

Preparation of PEGDMA Microparticles by a Polymer/Polymer Suspension.

PEGDMA 600 was dissolved in distilled water to produce a 50% (wt/wt) solution. DMPA photoinitiator (2% by monomer weight) was subsequently added. Dextran (Aldrich) of MW approximately 70,000 was added to distilled water and a 33% (wt/wt) stock solution was prepared. Aliquots of 2 g of aqueous monomer solution were added to 10 g of stock dextran solution and stirred at approximately 150 rpm to form dispersed monomer droplets.

Aliquots (2 g) of two-phase mixture were placed between two 50 mm×75 mm glass slides separated by Teflon® spacers of 0.9 mm thickness. The plates were irradiated with UV light (1 mW/cm²) for 20 minutes. Particles were twice washed with water and centrifuged (3,000× g) for 15 minutes to remove excess dextran. Particles were vacuum dried for 24 hours.

Preparation of PEGMA/PEGDMA Microparticles by a Solution/Precipitation Reaction.

Poly(ethylene glycol monomethacrylate) (PEGMA) (Polysciences, Inc., Warrington, Pa.) and PEGDMA microparticles were prepared by a modified dispersion polymerization technique. PEGMA (500 mg) and PEGDMA (500 mg) were dissolved in 30 g distilled water along with 25 mg Irgacure® 184 (Ciba-Geigy, Hawthorne, N.Y.) as photoinitiator. The solution was stirred at 300 rpm in a 100 ml glass beaker and irradiated with UV light (1 mW/cm²) for 30 minutes. The cloudy suspension was twice centrifuged (3,000× g) for 15 minutes and was resuspended with distilled water. Particles were dried in a vacuum oven for 24 hours.

Preparation of Microparticles of Poly(methacrylic Acid) by Solution/Precipitation Polymerization.

Methacrylic Acid (MAA) (Aldrich) was first vacuum distilled to remove the inhibitor p-methoxyphenol. MAA (250 mg), tetraethylene glycol dimethacrylate (TEGDMA) (Polysciences) (30 mg) and lrgacure® 184 (2.5 mg) as photoinitiator was added to 3 g distilled water. The solution was placed in a 15 ml brown glass vial and irradiated with UV light (1 mW/cm²) for 10 minutes. Particles were centrifuged (3,000× g) and resuspended twice in water to remove any freely soluble polymer. Particles were vacuum dried for 24 hours.

Preparation of Microparticles of Poly(2-hydroxyethylmethacrylate) by Solution/Precipitation Polymerization.

Microparticles of 2-hydroxyethyl methacrylate (HEMA) (Aldrich) were prepared by a similar solution/precipitation polymerization. HEMA was first vacuum distilled to remove free radical inhibitors. HEMA (250 mg) was dissolved in 3.5 g distilled water along with 19 mg TEGDMA crosslinking agent and 5 mg Irgacure® 184 as photoinitiator. This monomer solution was placed in a 15 ml brown glass vial and irradiated with UV light (1 mW/cm²) for 10 minutes. Resulting particles were centrifuged and washed with water as previously defined.

Preparation of Microparticles of Poly(Acrylic Acid) by Solution/Precipitation Polymerization.

Acrylic Acid (AA) (Aldrich) was first uninhibited by passing through a uninhibiting column (Polysciences) to remove free radical inhibitors. A stock solution for solution/precipitation polymerizations was prepared from 1 g AA, 40 mg Irgacure® 184 as photoinitiator, 60 mg TEGDMA, and 15 g distilled water. Aliquots of 2 ml were removed and polymerized in a 15 ml brown glass vial for varying amounts of time. All aliquots were irradiated with 1 mW/cm² UV light.

### EXAMPLE 2

### Characterization of Microparticles

Characterization of Microparticles by Optical Microscopy.

A Microstar One-Ten (AOO Scientific Inc, Buffalo, N.Y.) optical microscope equipped with video camera was used to determine particle size distribution. A sample of particles was counted (n=100) and each diameter was recorded. Minimum size of particles able to be sized by this method was approximately 1 μm.

Determination of Microparticle Particle Size.

A Coulter Counter® Multisizer IIe (Coulter Electronics, Hialeah, Fla.) was used to size particles automatically. A 70 μm aperture, useful for sizing particles in the range of 1.4-42 μm, was used for all samples. Particles were first suspended in Isoton® II (Beckman-Coulter, Inc., Fullerton, Calif.) saline buffer.

Characterization of Microparticles by Scanning Electron Microscopy.

A Jeol JSM-35CF Scanning Electron Microscope (Peabody, Mass.) was used to obtain micrographs of microparticles. Samples were mounted on carbon tape after lyophilization and sputter-coated with gold using a Hummer 6.2 Sputtering System (Anatech, LTD, Alexandria, Va.).

Results and Discussion

Preparation of PEG Containing Microparticles by Free Radical Polymerization of PEG Macromonomers.

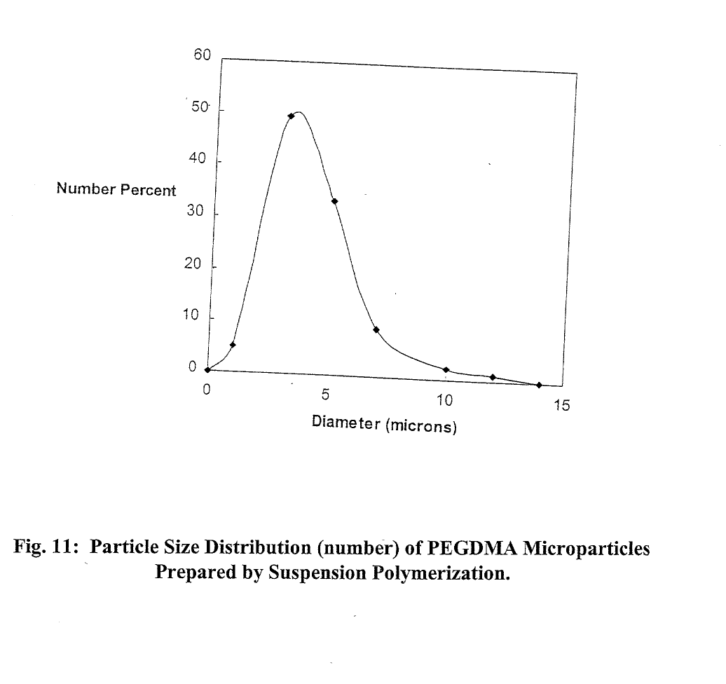

Three techniques were used to prepare microparticles incorporating large amounts of PEG by the polymerization of PEGMA and PEGDMA. The first of which was a conventional suspension polymerization. Typically, a surfactant pair is used to adjust for the correct degree of hydrophobicity and hydrophilicity necessary to form a stable emulsion or semistable suspension. This is often referred to as the Hydrophile-Lipophile Balance (HLB). One pair used often in inverse emulsions is Tween® 80 (poly(oxyethylene) sorbitan ester) and Span® 80 (sorbitan monooleate). Tween® 80 was not used because PEGMA and PEGDMA were thought to stabilize the system as well as Tween® 80. Silicon oil with 1% (wt/wt) sorbitan monooleate alone was used for the production of microparticles by suspension polymerization. Microparticles produced by this technique were found to have a number average diameter of 4 μm as determined by optical microscopy and were slightly polydisperse (FIG. 11). Suspension polymerization in the absence of sorbitan monooleate was also tried, but no microparticles were formed.

A similar suspension polymerization of PEGDMA was also attempted except thermal initiation was used instead of UV light. Reaction temperature was chosen as 70° C. Although the system showed initial phase separation at room temperature, heating to 70° C. diminished the stability of the two-phase system and prevented a suspension polymerization at that temperature.

The second technique used to produce microparticles of PEGDMA was by a polymer/polymer suspension. Thermodynamic data for the phase separation of PEG and dextran are readily available for systems of relatively high PEG molecular weight. The PEGDMA macromonomer used in this study had an average PEG chain molecular weight of 600. Thus, thermodynamic data available in the literature were not applicable. It was found that significantly more concentrated solutions of PEG and dextran were needed to cause phase separation. A stock solution of 33% (wt/wt) dextran was used in the experiments and 50% (wt/wt) solutions of PEGDMA were added.

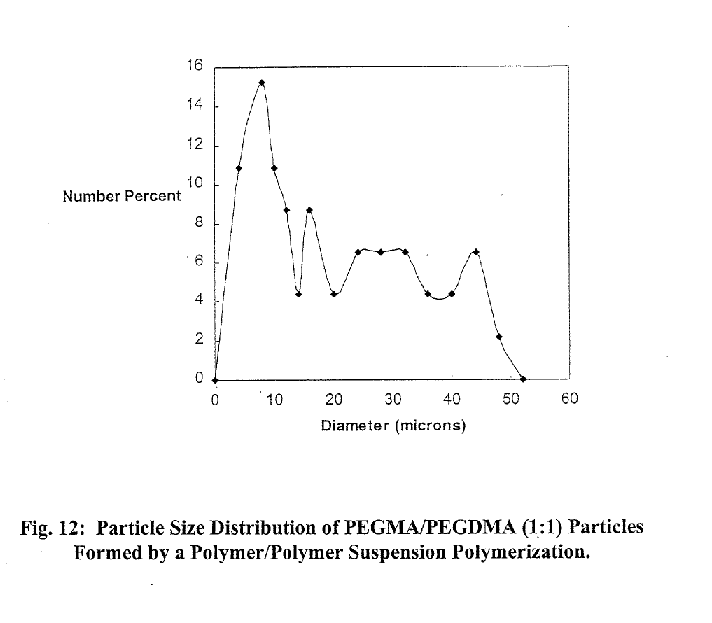

The PEGDMA/dextran two-phase mixture formed microparticles upon irradiation. Because of the low surface tension of the two phases, phase separation was stable for several minutes. After forming dispersed droplets rich in PEGDMA, aliquots were removed and placed between glass cover slides for irradiation. Following polymerization, microparticles were part of a viscous gel that could be broken by addition of excess water. Particle size distribution was very broad (FIG. 12) with a mean particle diameter (number average) of 20 μm. It is hypothesized that the lack of mixing allowed forming particles to coalesce prior to hardening leading to the polydispersity which lead to the broad distribution seen in FIG. 12.

Attempts at polymerizing more than 5 g of two-phase mixture with agitation resulted in the formation of a solid polymer mass. Compositions were identical as for those microparticles prepared by polymerization between glass slides. As seen in the water-in-oil suspension polymerization, microparticles may be produced in an agitated two-phase system initiated by UV light. Although it is likely that much of the UV light is scattered by the two-phase system, only a small penetration depth is needed because over time stirring will bring all droplets near the surface. From this observation, it is submitted that quick reaction of the monomer droplet is key to production of particles by the polymer/polymer suspension method.

A third method for the production of microparticles of PEGMA and PEGDMA was by a modified dispersion polymerization. In conventional dispersion polymerizations, reaction solvent is key to production of particles. A liquid must be chosen such that it dissolves monomer, initiator, and any suspending agent. This liquid, however, must be a poor solvent for the resulting polymer. As the polymer chains grow, they aggregate and form a second phase. This eliminates the need for a continuous phase, which may be difficult to remove during recovery of the particles. In the PEGMA/PEGDMA system, the monomer and ensuing polymer are both soluble in the reaction medium. However, it was proposed that crosslinks would cause growing polymer chains to precipitate out as a second phase after a critical threshold had been reached.

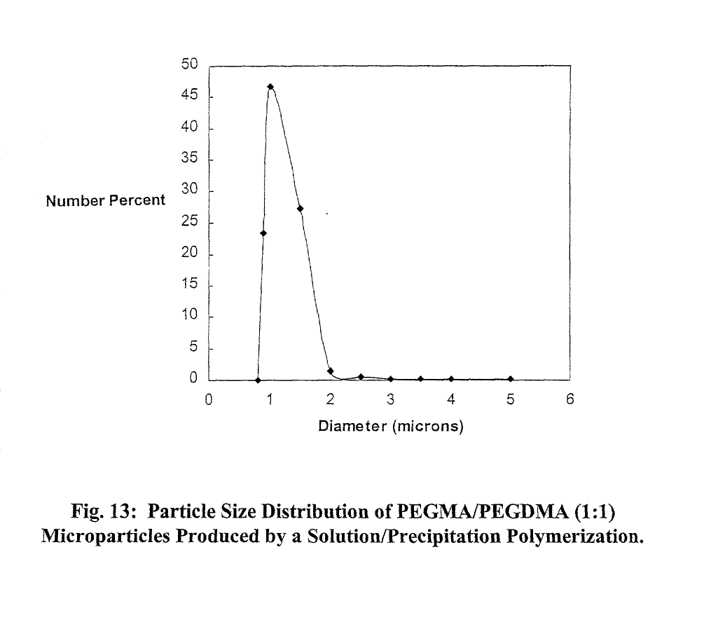

Production of particles using this technique yielded microparticles with a low degree of polydispersity (FIG. 13). The mean diameter (number average) was around 1.2 μm as determined by optical microscope. Varying the initiator concentration had little effect on the size of the microparticles. Also, increasing the initial concentration of monomer did not significantly change particle size.

Preparation of Microparticles of Poly(Methacrylic Acid)

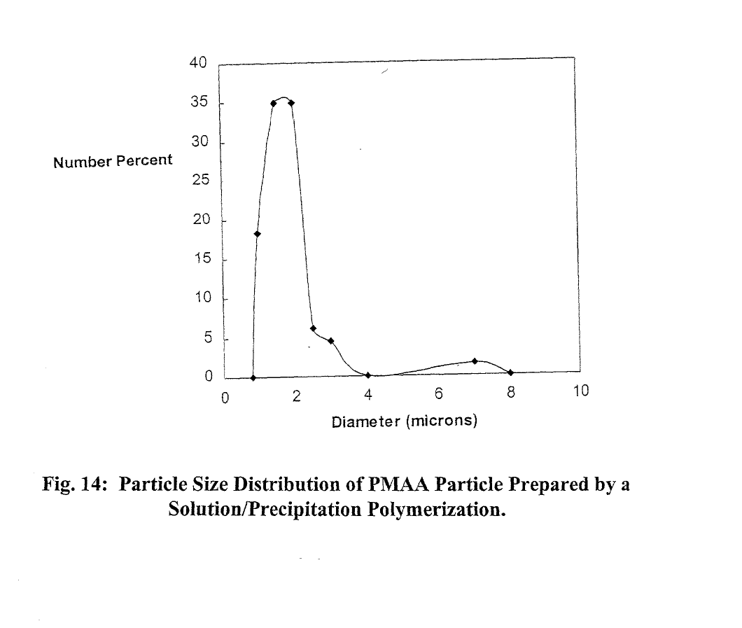

Microparticles of PMAA were prepared by a solution/precipitation polymerization. The prepared particles were monodisperse (FIG. 14) with a mean particle diameter of 1 μm as determined by optical microscopy. A glassy polymer residue was also found on the bottom of the polymerization vial. Particles agglomerated into 2-3 particle clusters while in solution. Sonication dispersed the particles temporarily.

Preparation of Microparticles of Poly(2-hydroxyethyl Methacrylate).

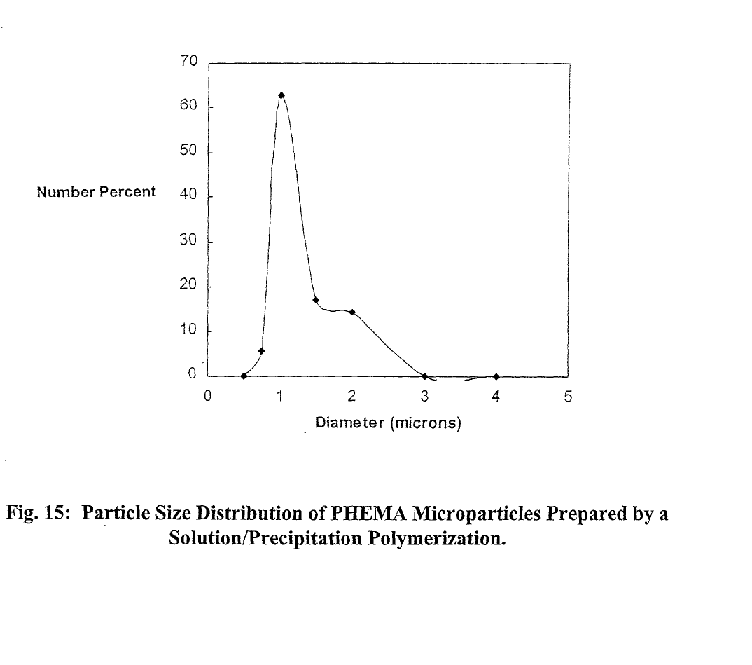

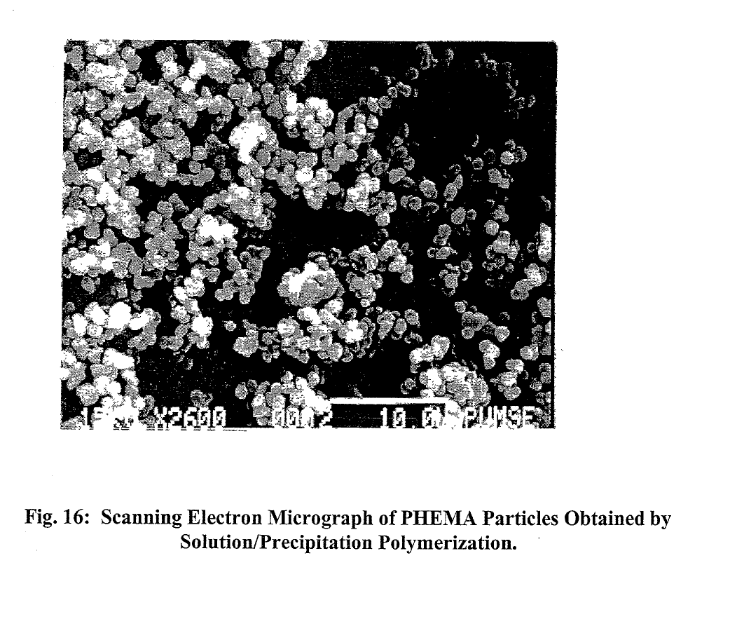

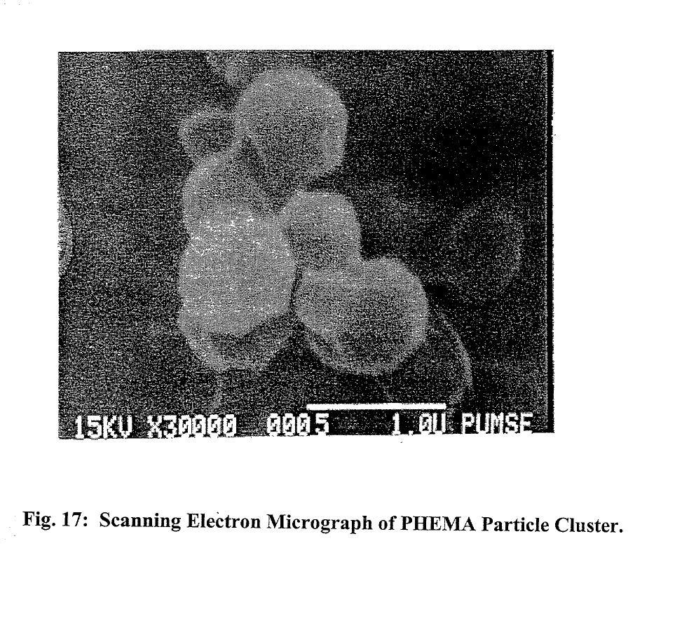

A solution/precipitation polymerization was used to produce microparticles of PHEMA. The particle size distribution was narrow with a mean particle diameter (number average) of 1 μm with some particles also in the size range of 2 μm (FIG. 15). Scanning electron micrographs were obtained (FIGS. 16 and 17) to study the morphology of the resulting polymer microparticles. As shown in FIG. 16, microparticles are nearly monodisperse and aggregate extensively. Like particles of PMAA, sonication temporarily dispersed the particle clusters. While swollen, particles appear spherical. However, after freeze drying particles appear to collapse (FIG. 17).

Proposed Mechanism of Solution/Precipitation Polymerization.

In conventional dispersion polymerizations, increasing the concentration of initiator decreases the size of the particles. The initial number of free radicals produced determines the largest possible number of particles. As the number of particles grows, there are more sites for aggregation of growing polymer chains and the ultimate particle volume is smaller. Similarly, increasing the initial charge of the monomer increases the diameter of particles in a typical dispersion polymerization. These trends were not seen in the solution/precipitation polymerization and are indicative of a mechanism different than that for conventional dispersion polymerization. Because of the monodispersity of the system and the independence of the particle size distribution from initial monomer and initiator concentration a self-associating mechanism is proposed.

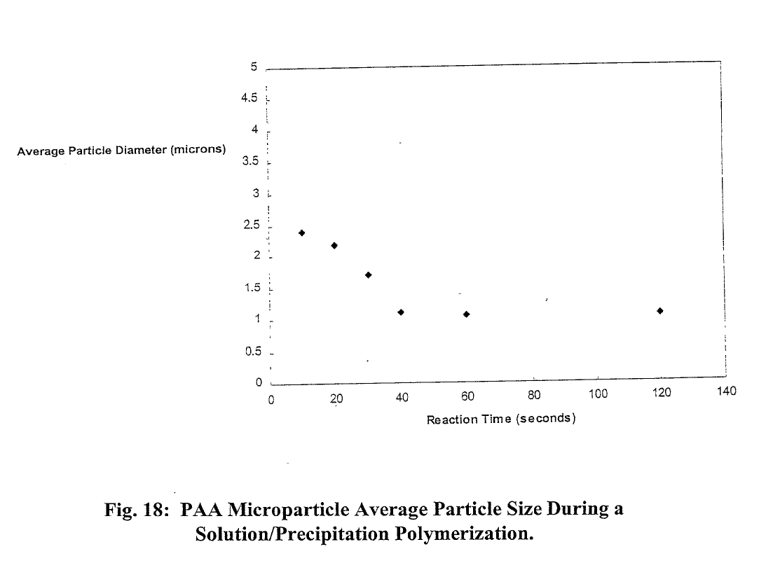

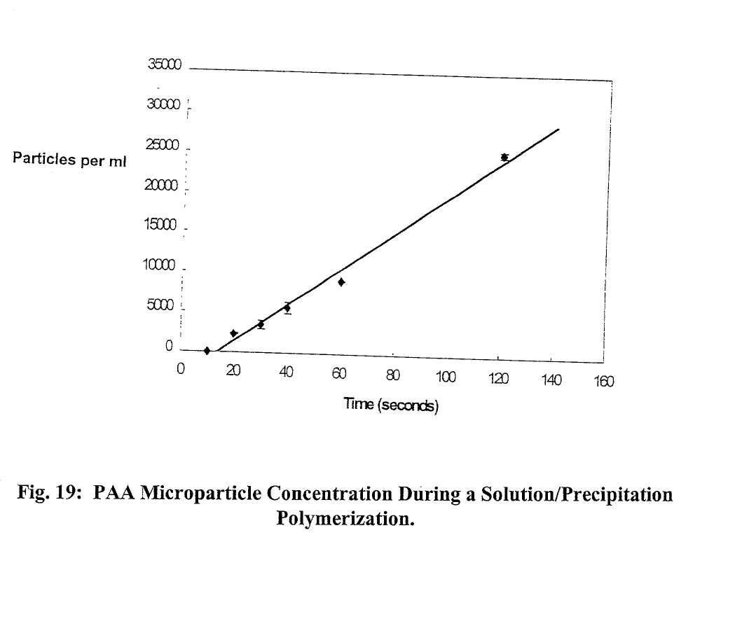

Cumulative particle size distribution and particle concentration were recorded during a solution/precipitation to help elucidate the polymerization mechanism. A solution/precipitation polymerization of acrylic acid was chosen since the resulting particles do not agglomerate and are large enough to be sized by optical microscope. Mean particle diameter versus reaction time (FIG. 18) shows that particle size differs during the course of the reaction. The initial particles produced in the reaction have an average diameter of 2.4 μm. Particle formation begins after nearly 20 seconds and particle concentration increases linearly (FIG. 19) for the first two minutes. As the reaction proceeds, instantaneous microparticle size decreases slightly until an equilibrium value is reached and nearly monodisperse particles of average diameter 1.0 μm are produced until the reaction is completed. It is hypothesized that in the first part of the reaction, which is defined by no particle formation, a solution polymerization is occurring. After a critical threshold is reached the growing polymer chains associate and particle formation begins.

Conclusions

Microparticles of various hydrophilic polymers were successfully prepared by several techniques and subsequently characterized. A conventional suspension polymerization, an all-aqueous polymer/polymer suspension polymerization, and a modified dispersion polymerization were used to prepare microparticles of PEGMA/PEGDMA with mean particle diameters of 4 μm, 20 μm, and 1.2 μm respectively. Microparticles of PMAA, PAA, and PHEMA were also produced by a modified dispersion polymerization and all reactions resulted in mean particle diameters of 1 μm.

The kinetics of the modified dispersion polymerization reaction were also studied. The first part of the reaction is characterized by a period of no particle formation. After this critical time, particle formation is rapid and particle concentration increases linearly for the next two minutes of the reaction. Initially formed particles are slightly larger than those formed in the latter part of the reaction and are more polydisperse.

### EXAMPLE 3

### Preparation of PAA and P(AA-g-PEG) Microparticles

Preparation of Microparticles

The microparticles were prepared using a toxic-solvent-free method of synthesis of poly(acrylic acid)-based microparticles by free radical ‘dispersion’ polymerization. The acrylic acid (AA, Aldrich, Milwaukee, Wis.) monomer was vacuum distilled prior to use in order to remove a methoxyphenol inhibitor. The poly(ethylene glycol) acrylate 300 (PEGA 300, Aldrich, Milwaukee, Wis.) monomer was used as received. Tetraethyleneglycol diacrylate (TEGDA, Polysciences, Warrington, Pa.) was used as a crosslinking agent and a 0.5 mM aqueous solution of sodium dodecyl sulfate (SDS, Sigma, St. Louis, Mo.) was used as the outer phase. To initiate the reactions, 1-Hydroxy cyclohexyl phenyl ketone (Irgacure® 184, Ciba, Tarrytown, N.Y.) was utilized as a UV initiator.

In a typical experiment, 0.030 g of Irgacure® 184 was dissolved in 1 g of AA in an amber glass vial followed by the addition of 0.188 g of PEGA 300 and 0.316 g of TEGDA. This monomer mixture was added dropwise to 56.143 g of a 0.5 mM aqueous solution of SDS, which was agitated using a magnetic stir bar. The solution was exposed to UV light at an intensity of 3 mW/cm² for 45 minutes. The particles were then washed by transferring the resulting dispersion in regenerated cellulose membranes, which were immersed in deionized water for 5 days changing the water twice daily.

Microparticle Characterization

The particle size distributions of the washed particles were analyzed by photon correlation spectroscopy (PCS) using Coulter® N4 Plus Submicron Particle Sizer (Coulter, Miami, Fla.).

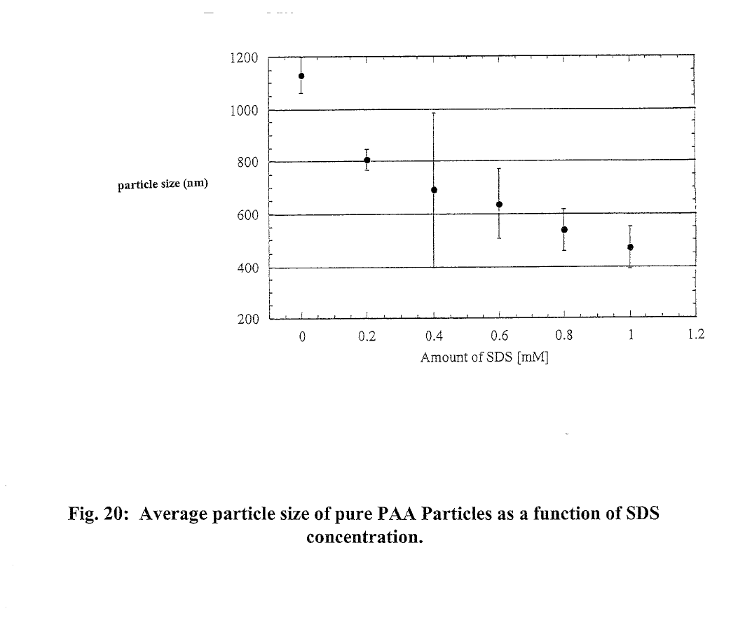

The effect of the surfactant, SDS, on the average particle size of pure cross-linked PAA particles is shown in FIG. 20. The crosslinking ratio in these experiments was kept constant at 0.11 moles of crosslinker per total moles of monomers and the total concentration of monomers was kept at 1.75 wt %. The average particle size was inversely proportional to the amount of the surfactant in the concentration range below the critical micelle concentration of SDS, which was determined to be 8.1 mM at 25° C. It is important to notice that PAA particles in the range of 1,100 nm were produced even in the absence of any surfactant.

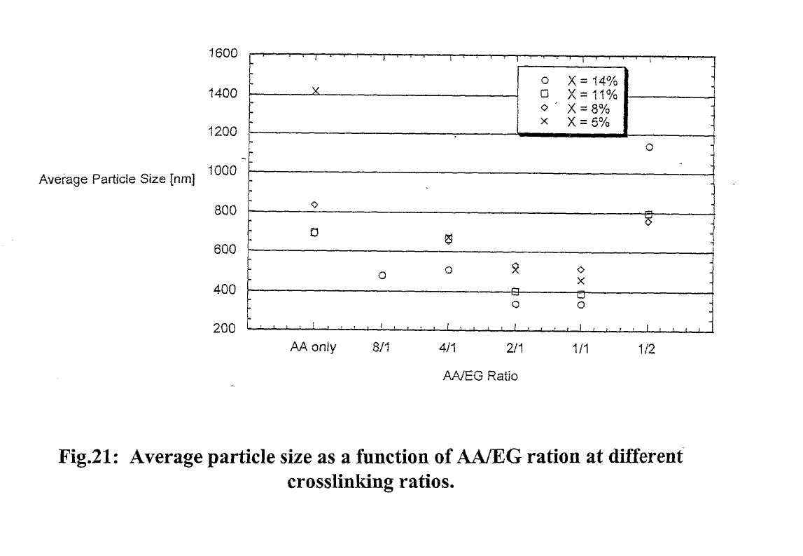

FIG. 21 shows the effect of degree of crosslinking and AA/EG ratio on the average particle size after washing. Clearly, there is a minimum microparticle size as a function of EG content at any degree of crosslinking. Furthermore, crosslinking offers an additional means of controlling the particle size at a particular AA/EG ratio.

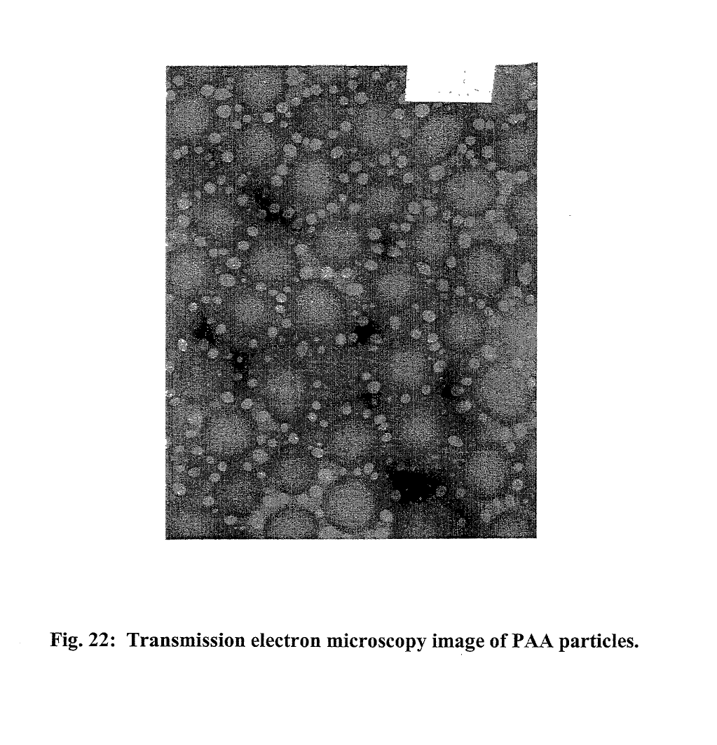

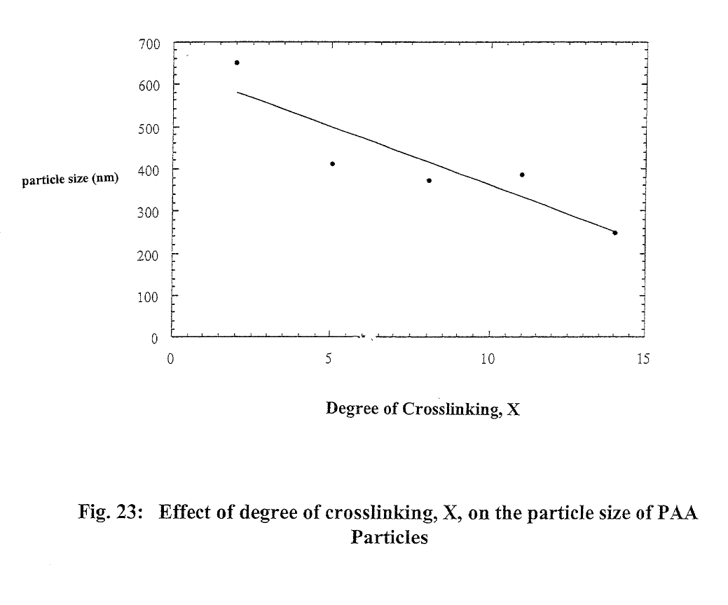

In order to confirm the results of particle size distributions obtained from the light scattering technique, a complimentary sizing method was used. In the size range of interest, transmission electron microscopy (TEM) is the second most widely used technique to evaluate the average particle size of an ensemble of particles. In addition to being an absolute technique for measuring particle size, it allows for direct observation of the morphology of the particles. FIG. 22 is a TEM image of pure PAA particles whose average particle size was estimated to be 1,100 nm using PCS. However, the average particle size obtained using TEM was 56 nm. This difference most likely results from the very low mechanical integrity of the prepared particles as indicated by the collapse of the particles due to the evaporation of the aqueous media in the vacuum environment in the microscope. In order to verify this hypothesis, a metal shadowing experiment was conducted using TEM. This type of experiment is commonly used to elucidate the shape of a specimen by evaporating a thin heavy metal coating (Au in our case) at an oblique angle and observing the resulting shadow shape. The fact that we could not observe any shadows confirmed the collapse of the particles upon exposure to the low pressure of the sample chamber in the TEM. The observed mechanical properties of the particles are most likely due to an insufficient incorporation of the crosslinking agent into the structure of the polymer network.

It has been shown that the formation of hydrogen-bonded complexes affects the reaction kinetics of acrylic and methacrylic acid. It is also known that the formation of hydrogen bonding complexes occurs only below a critical pH value. Above this critical pH there is not a sufficient number of protonated carboxylic acid groups for the formation of hydrogen bonds. Therefore, to investigate the possible effect of hydrogen bonding on the formation of particles, a series of five aqueous polymerization solutions was prepared and the pH of the solutions was adjusted by the addition of 0.2 N sodium hydroxide to 2.5, 3.0, 3.5, 4.0 and 4.5, respectively. The effect of environmental pH on the formation of particles of P(AA-g-PEG) is summarized in Table 1.

Clearly, the pH of the polymerization solution has a pronounced effect on the formation of particles. The pKa of a weak acid is defined as the pH at which half of the acidic groups are ionized while the other half remains protonated. The pKa of the AA monomer is 4.25.

The experimental data suggest that there exists a minimum in the amount of protonated carboxylic groups needed for the formation of particles since the critical pH at which particle formation takes place increases with increasing AA content in the monomer solution. Due to the well known relationship between pH and hydrogen-bond formation, this data suggests that the formation of hydrogen bonds might be critical in the particle formation mechanism.

To further confirm the hypothesis that the formation of hydrogen-bonded complexes between the acrylic acid and PEG-containing macromonomer plays a significant role in the particle formation mechanism, the polymerization reactions were carried out in mixtures of ethanol and water. Ethanol is an aprotic solvent that is known to disrupt the formation of hydrogen-bonded complexes. Mixtures of ethanol and water in weight ratios of 100:0, 75:25, 50:50, 25:75 and 5:95 were used. The effect of increasing the amount of ethanol in the solvent mixture is illustrated in Table 2. Clearly, the amount of an H⁺ donating solvent such as ethanol has a profound effect on the formation of the particles.

Proposed Mechanism of Microparticle Formation

The aforementioned experiments have led to a hypothesized mechanism of particle formation. Initially, the monomers, initiator and cross-linker are dissolved in the solvent. The polymerization reaction is initiated by exposing the mixture to the UV-light that breaks each initiator molecule into two active radicals. These radicals react with the water-soluble monomers forming oligomer radicals. The incorporation of PEG macromonomers is most likely fairly limited due to ‘shielding’ of the acrylate reactive group by the long polyethylene glycol chain. The extent of shielding increases with a decreasing compatibility of the solvent with the PEG chain and is the highest in a poor solvent in which the long PEG chain coils up around the reactive group. During the third stage of the reaction, hydrogen-bonded complexes are formed between the carboxylic acid groups on the oligomer radicals and ether oxygens on the PEGA macromonomers. Each network of the oligomer radicals connected via hydrogen bonds functions as a precursor for a mature particle. This nucleation period continues to take place until there is a sufficient number of particles such that the new oligomer radicals and PEG macromonomers enter the already existing particles instead of forming new ones. During the fourth growth stage of the process the particles grow as the AA and cross-linker monomers, and PEG macromonomers are continuously being incorporated into the polymer network. The particle growth stops once all the growing chains are terminated or all of the monomers are depleted from the solution.

TABLE 1
Summary of the effect of pH of the
reaction media on formation of particles

| pH | AA/EG = 4.0 | AA/EG = 2.0 | AA/EG = 1.0 |
|---|---|---|---|
| 2.50 | Yes | Yes | Yes |
| 3.00 | Yes | Yes | Yes |
| 3.50 | Yes | Yes | Yes |
| 4.00 | Yes | Yes | No |
| 4.50 | No | No | No |

TABLE 2
Summary of the effect of amount of ethanol in the solvent mixture

| EtOH [wt %] | 5 | 25 | 50 | 75 | 100 |
|---|---|---|---|---|---|
| AA/EG = 2.0 | Yes | No | No | No | No |

### EXAMPLE 4

### Preparation of PAA-based Gels

Preparation of Microparticles

The procedures were as described in Example 3, except that poly(ethylene glycol)-containing methacrylates were used rather than poly(ethylene glycol) acrylate 300 (i.e., PEGA 375 was used rather than PEGA 300).

Particle Size Determination

The procedures were as described in Example 3. FIG. 23 shows the effect of degree of crosslinking on the average particle size of PAA particles. As a result of higher crosslinking of the particles, the average particle size decreases. Since at the present time there is no single experimental technique that is capable of confirming the degree of incorporation of the crosslinking agent in the particles, one can merely acknowledge that crosslinking offers additional means of controlling the particle size. Similar observations were made for P(AA-g-PEG) particles at various ratios of AA/EG.

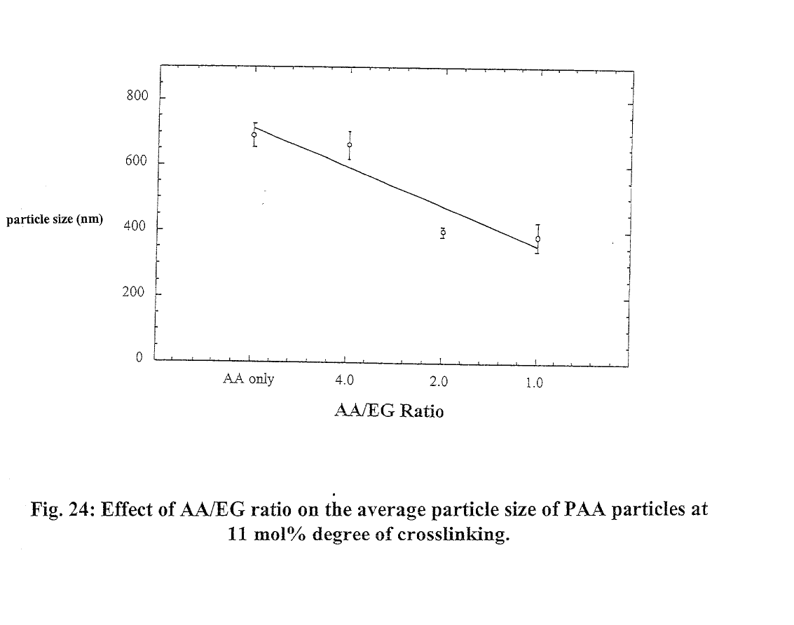

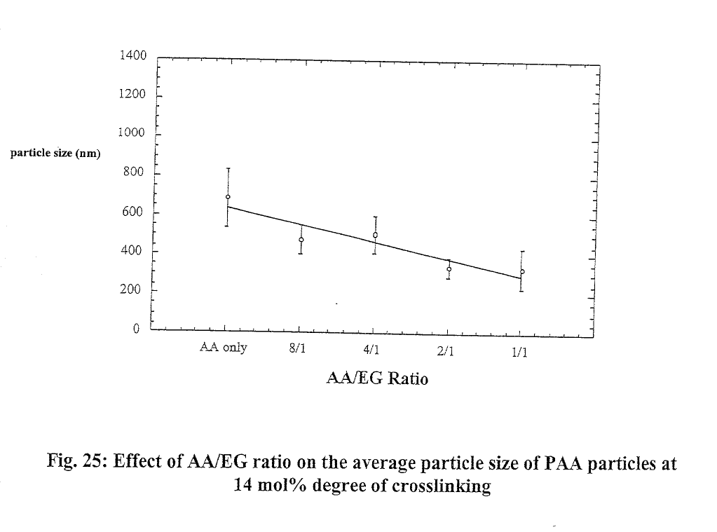

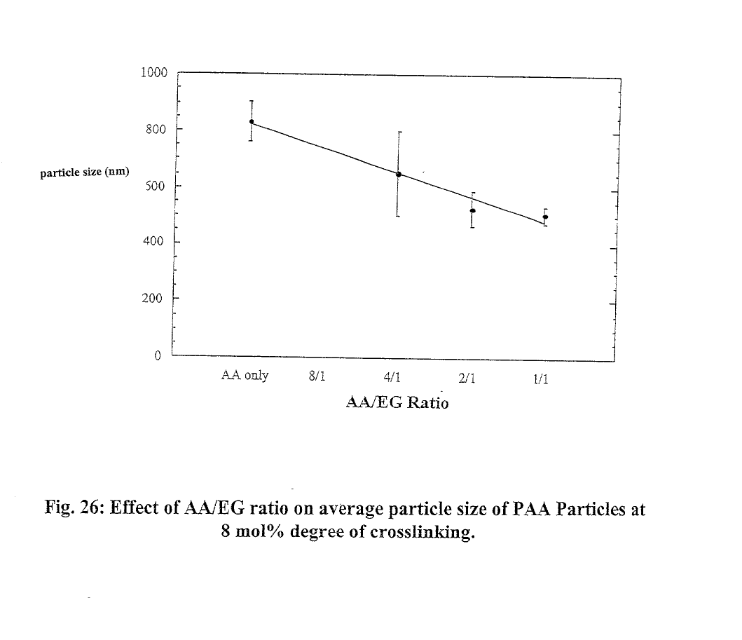

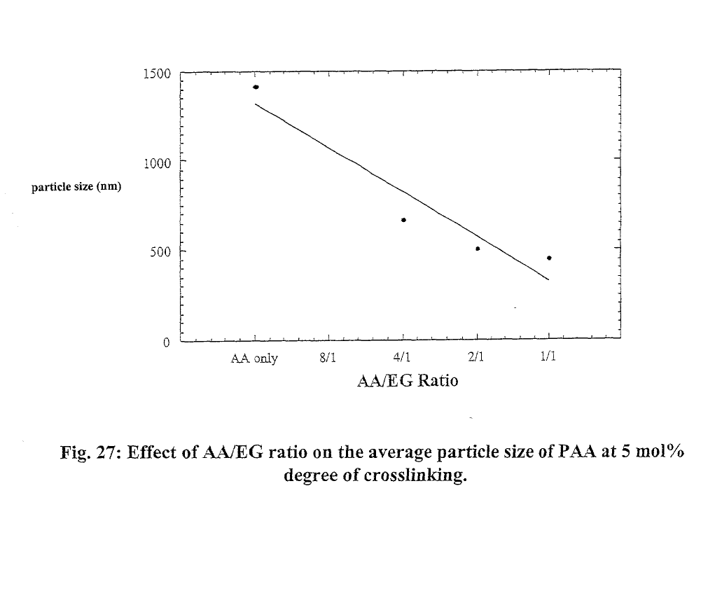

FIGS. 24-27 show the effect of AA/EG ratio on the particle size at crosslinking ratios between 14 and 5 mol %. Regardless of the degree of crosslinking, particle size decreases with increasing amount of the PEG-containing methacrylate in the polymerization mixture. There are several possible reasons for the observed trend. First, this trend might be the result of decreased reactivity of the methacrylate group which in turn would cause fewer monomers to be incorporated into the particle. Second, the PEG-containing methacrylates might function as additional emulsifiers during the polymerization process. Thus, increasing the number of PEG-containing moieties would result in a larger number of particles that would be smaller in size. Third, the fairly large PEG-containing methacrylates cannot enter the particles during their growth stage due to the SDS molecules adsorbed on the outer surface of the particles. Therefore, the SDS molecules would effectively prevent the entrance of comonomers based on their size. This explanation is consistent with the observation of the effect of SDS on the average particle size of pure PAA particles (see FIG. 20).

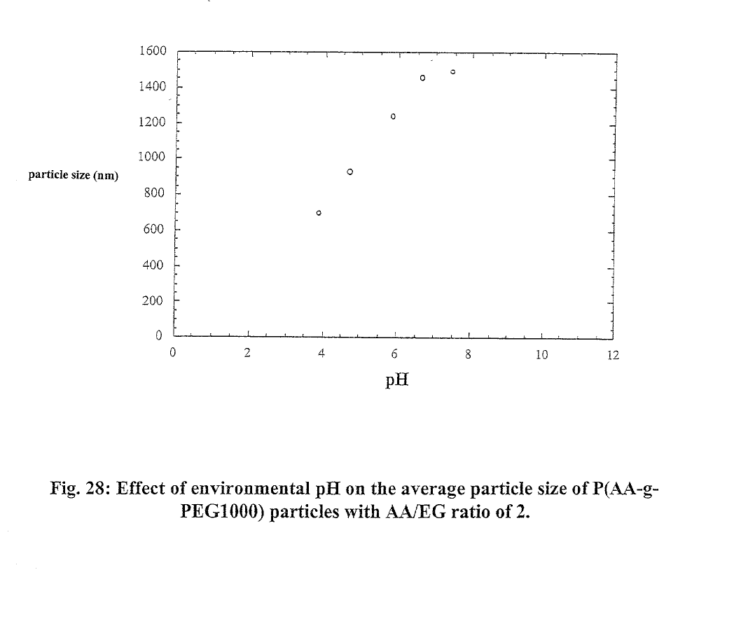

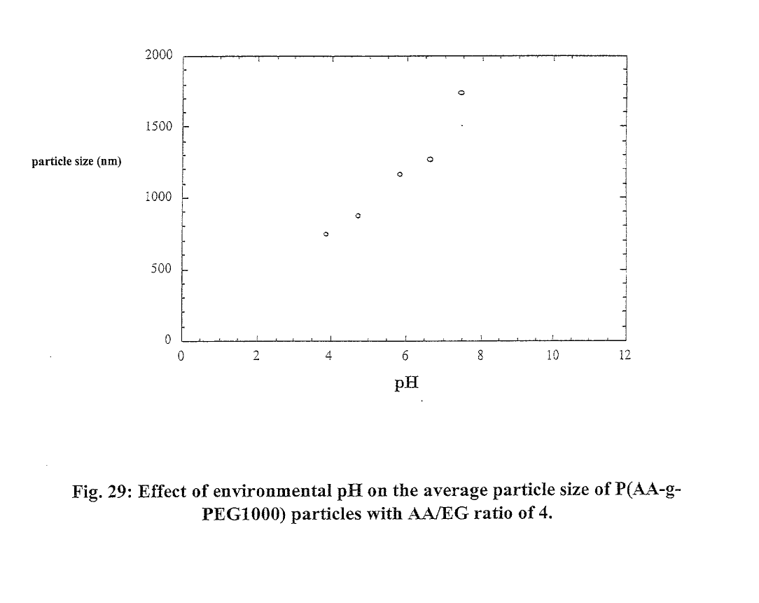

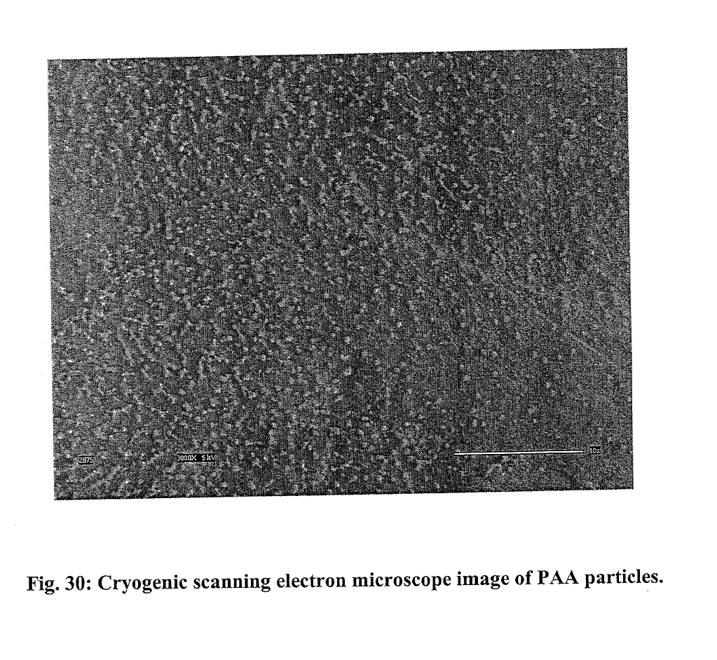

FIGS. 28 and 29 show the effect of pH on particle size. With increasing pH particle size increases.

Cryogenic Scanning Electron Microscopy

To investigate the particles in their most natural wet state, cryogenic scanning electron microscopy (SEM) was performed. A JEOL JSM-840 SEM equipped with a cooling stage was used to conduct the cryogenic SEM studies. Prior to taking an image of the sample, the liquid dispersion of the particles was placed on a copper grid which was then placed inside of a temperature controlled stage where the temperature was maintained at −140° C. The sample was quenched by exposure to this environment. In order to better reveal the details of the sample in the SEM, the sample was plasma-coated prior to transferring it to the cold stage of the SEM. FIG. 30 shows an image of PAA particles obtained with a cryogenic SEM. One can clearly see the spherical shape of the submicron particles. Occasionally, a cluster of particles is also noticed which is most likely the result of moisture among several particles being frozen during the initial quenching of the sample. The results of cryogenic SEM support the data from PCS that the particles are on the order of approximately 0.8 microns in their wet state.

Proposed Mechanism of Particle Formation

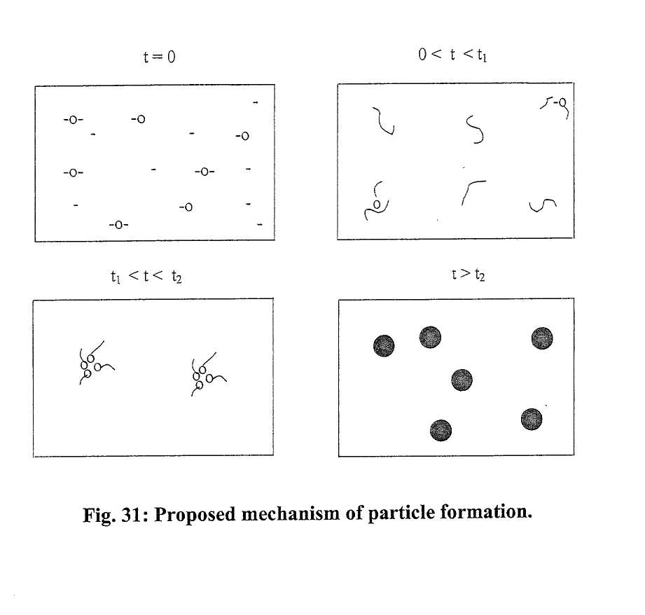

The aforementioned experiments have led to a formulation of a possible mechanism of particle formation. FIG. 31 illustrates the various stages during the formation of the particles. Initially, the monomers, initiator and cross-linker are dissolved in the solvent. The polymerization reaction is initiated by exposing the mixture to the UV-light, this instant is taken as time zero. During the stage that follows, mainly the AA monomers are able to react with each other thus creating oligomers of acrylic acid. It is possible that some of PEG macromonomers are also incorporated into the AA oligomers, even though their incorporation is most likely fairly limited due to ‘shielding’ of the methacrylate reactive group by the long polyethylene glycol chain. The extent of shielding increases with a decreasing compatibility of the solvent with the PEG chain and is the highest in a poor solvent in which the long PEG chain coils up around the reactive group. During the third stage of the reaction, hydrogen-bonded complexes between the carboxylic acid groups on the PAA oligomers and ether oxygens on the PEG chains are formed thus creating a network of the PAA oligomers. The polymerization reaction continues to take place both inside and outside of the networks until there is a sufficient number of particles that new oligomer chains and PEG macromonomers become part of existing particles instead of forming new ones. During the fourth stage of the process the particles grow as the cross-linker and PEG macromonomers are continuously being incorporated into the polymer network. The particle growth stops once all the growing chains are terminated or all of the monomers are depleted from the solution.

Conclusions

A toxic-solvent-free polymerization technique for the production of PAA-based networks in the micro- and nanometer range was developed using water as the outer phase. The average size of these particulates was assessed using light scattering, TEM and cryogenic SEM. Experimental results suggest that the mechanism of particle formation is precipitation and that the primary parameter to control the particle size is the concentration of SDS stabilizer in the aqueous phase.

What is claimed is:

1. A method of forming a crosslinked hydrogel in the form of substantially uniform microparticles said method comprising
polymerizing a monomer composition comprising a C₃-C₆ unsaturated carboxylic acid and a polyolefin crosslinking agent dispersed in an aqueous medium at a pH of less than 4.5.

2. The method of claim 1 wherein the monomer composition further comprises a polyalkylene glycol monoacrylate or monomethacrylate.

3. The method of claim 1 wherein the aqueous medium comprises a surfactant.

4. The method of claim 1 wherein the monomer composition further comprises a free radical initiator.

5. The method of claim 2 wherein the monomer composition further comprises a free radical initiator.

6. The method of claim 3 wherein the monomer composition further comprises a free radical initiator.

7. The method of claim 4 wherein the free radical initiator is heat activated.

8. The method of claim 5 wherein the free radical initiator is heat activated.

9. The method of claim 6 wherein the free radical initiator is heat activated.

10. The method of claim 4 wherein the free radical initiator is UV light activated.

11. The method of claim 5 wherein the free radical initiator is UV light activated.

12. The method of claim 6 wherein the free radical initiator is UV light activated.

13. The method of claim 7 wherein the free radical initiator is selected from peroxides and azobisisobutyrylnitrile.

14. The method of claim 8 wherein the free radical initiator is selected from peroxides and azobisisobutyrylnitrile.

15. The method of claim 9 wherein the free radical initiator is selected from peroxides and azobisisobutyrylnitrile.

16. The method of claim 1 wherein the polymerization is initiated by γ-radiation or electron beam radiation.

17. The method of claim 2 wherein the polymerization is initiated by γ-radiation or electron beam radiation.

18. The method of claim 3 wherein the polymerization is initiated by γ-radiation or electron beam radiation.

19. The method of claim 3 wherein the C₃-C₆ unsaturated carboxylic acid is acrylic acid and the surfactant is sodium dodecyl sulfate.

20. The method of claim 3 wherein the surfactant is a cationic surfactant.

21. The method of claim 3 wherein the surfactant is an anionic surfactant.

22. The method of claim 3 wherein the surfactant is a nonionic surfactant.

23. The method of claim 1 wherein the crosslinking a gent is di-olefin functional.

24. The method of claim 2 wherein the crosslinking agent is di-olefin functional.

25. The method of claim 3 wherein the crosslinking agent is di-olefin functional.

26. The method of claim 1 wherein the crosslinking agent is a polyalkyleneglycol diacrylate or dimethacrylate.

27. The method of claim 2 wherein the crosslinking agent is a polyalkyleneglycol diacrylate or dimethacrylate.

28. The method of claim 3 wherein the crosslinking agent is a polyalkyleneglycol diacrylate or dimethacrylate.

29. A crosslinked hydrogel composition in the form of substantially uniform microparticles, said hydrogel composition comprising a crosslinked polymer formed by free radical polymerization of olefin monomers comprising a C₃-C₆ unsaturated carboxylic acid and a water dispersible polyolefin crosslinking agent in an aqueous medium at a pH of less than about 4.5.

30. The hydrogel composition of claim 29 wherein the carboxylic acid is acrylic or methacrylic acid.

31. The hydrogel composition of claim 29 wherein the olefin monomers further comprise a polyalkyleneglycol monoacrylate or monomethacrylate.

32. The hydrogel composition of claim 31 wherein the polyalkylene glycol monoacrylate or monomethacrylate has a molecular weight of about 200 to about 2500.

33. The hydrogel composition of claim 26 wherein the microparticles have an average diameter of about 1 to about 1000 microns.

34. The hydrogel composition of claim 27 wherein the microparticles have an average diameter of about 1 to about 1000 microns.

35. The hydrogel composition of claim 28 wherein the microparticles have an average diameter of about 1 to about 1000 microns.

36. The hydrogel composition of claim 29 wherein the crosslinking agent is a polyalkyleneglycol diacrylate or dimethacrylate.

37. The hydrogel composition of claim 29 wherein the crosslinking agent is di-olefin functional.

38. The hydrogel composition of claim 29 wherein the crosslinking agent is tri-olefin functional.

39. The hydrogel composition of claim 26 wherein the microparticles have an average diameter of about 0.1 to about 500 microns.

40. The hydrogel composition of claim 27 wherein the microparticles have an average diameter of about 0.1 to about 500 microns.

41. The hydrogel composition of claim 28 wherein the microparticles have an average diameter of about 0.1 to about 500 microns.
